(19) United States
(12) Patent Application Publication
(10) Pub. No.: US 2019/0360135 A1
(43) Pub. Date: Nov. 28, 2019

(54) Sheet Of Loop Material, Apparatus And Method For Forming Same

(71) Applicant: 3M INNOVATIVE PROPERTIES COMPANY, St. Paul, MN (US)

(72) Inventors: Michael R. Berrigan, Oakdale, MN (US); Zackary J. Becker, St. Paul, MN (US); Daniel E. Johnson, Marine On St. Croix, MN (US); Jimmy M. Le, St. Paul, MN (US); John D. Stelter, Osceola, WI (US); Shou-Lu G. Wang, Duluth, GA (US); Robert C. Etter, Oakdale, MN (US)

(21) Appl. No.: 16/484,338

(22) PCT Filed: Feb. 9, 2018
(86) PCT No.: PCT/IB2018/050812

§ 371 Date: Aug. 7, 2019

Related U.S. Application Data
(60) Provisional application No. 62/458,760, filed on Feb. 14, 2017.

(51) Int. Cl. D04H 1/4374; B32B 7/05; B32B 5/02; D04H 1/56; D04H 3/03; A44B 18/00; A61F 13/62

(52) U.S. Cl. CPC D04H 1/4374; B32B 7/05; B32B 5/022; A61F 13/627; D04H 3/03; A44B 18/0011; D04H 1/56

(57) ABSTRACT

A web of nonwoven fabric suitable for, e.g., the loop portion of a hook-and-loop fastening system. The method for making this material relies on differential shrinkage of different layers to cause the loops to self-form. The method is robust and simpler than that previously used for similar constructions.

### TECHNICAL FIELD

The present disclosure relates a sheet of loop material adapted to be cut into pieces to form loop portions for fasteners of the type comprising releasably engageable hook and loop portions, to be incorporated into items such as disposable garments or diapers.

### BACKGROUND

Many sheets of loop materials are known that are adapted to be cut into pieces to form the loop portions for fasteners of the type comprising releasably engageable hook and loop portions. Such sheets of loop materials typically comprise a backing and a multiplicity of loops formed from longitudinally oriented polymeric fibers anchored in the backing and projecting from a front surface of the backing so that they may be releasably engaged with the hooks on the hook portion of such a fastener, and can be made by many methods including conventional weaving, or knitting techniques. Sheets of loop materials in which the loops are stitched into the backing are described in U.S. Pat. Nos. 4,609,581 and 4,770,917. U.S. Pat. No. 5,616,394 describes a sheet of loop material adapted to be cut into pieces to form loop portions for fasteners, which sheet of loop material includes a backing comprising a thermoplastic backing layer with generally uniform morphology, and a sheet of longitudinally oriented fibers having generally non-deformed, anchor portions bonded or fused in the thermoplastic backing layer at spaced bonding locations, and arcuate portions projecting from a front surface of the backing between the bonding locations.

While the loop fastener portions made from many such sheets of loop materials work well with many different hook fastener portions, many of the processes by which the sheets of loop material are made are more expensive than may be desired, particularly when the loop fastener portions are intended for a limited amount of use, such as to attach a disposable diaper to a person.

### SUMMARY

The present disclosure provides nonwoven fabrics including at least one spunbond web that can self-form loops because they have a shrinkage mismatch. This can be due to the raw materials chosen or the process conditions chosen so that the two webs differ in molecular orientation so that when exposed to latent heat one of the layers will shrink more than the other. Bonding the layers together before allowing latent heat to shrink the highly oriented web forces it to bow away from the other layer, forming loops. These loops can engage with hook constructions in a hook-and-loop fastener arrangement.

In one aspect, the present disclosure provides a method of forming a nonwoven fabric, comprising providing a first layer comprising continuous or discontinuous oriented monocomponent thermoplastic fibers; providing a second layer; conveying the first and second layers through a nip comprising a first and a second roller wherein the first roller is a patterned roller, wherein the nip pattern creates bonded regions while leaving unbonded regions so as to bond the first and second layers into a nonwoven fabric, wherein the unbonded regions comprise between about 20% and 40% of the total area of the nonwoven fabric, and further wherein the nip introduces latent heat into the nonwoven fabric; and allowing the latent heat to cool while the first layer is held at a tension of less than 4 N/linear cm such that free loops are formed in the unbonded areas of the first layer.

In some convenient embodiments, the continuous or discontinuous oriented monocomponent thermoplastic fibers within the first layer are spunbond polypropylene formed at a spinning speed of lower than 2400 m/min.

In some convenient embodiments, the first and second layers are each formed of webs of indefinite length having a longitudinal direction and a cross direction perpendicular to the longitudinal direction. In some of these embodiments, it is advantageous to arrange the pattern of bonding between the two webs such that no bonded regions extend without interruption by unbonded regions in any line oriented in the cross direction.

In some embodiments, in is convenient for the second layer further include a meltblown layer laminated to the continuous or discontinuous oriented monocomponent thermoplastic fibers. In some of these embodiments, the meltblown layer is disposed between two spunbonded layers.

In a second aspect, the present disclosure provides a nonwoven fabric, comprising a first layer comprising continuous or discontinuous oriented uncrimped monocomponent thermoplastic fibers, and a second layer having a pattern of bonded regions and unbonded regions with the first layer, wherein the bonded regions comprise between about 20% and 40% of the total area of the nonwoven fabric, wherein the first layer displays raised loops in the unbonded areas, and wherein the first layer possesses a percent solidity of less than 12.

In some convenient embodiments, the first and second layer comprise webs of indefinite length having a longitudinal direction and a cross direction perpendicular to the longitudinal direction. In these embodiments, it may be advantageous to arrange the pattern of bonding between the two webs such that no bonded regions extend without interruption by unbonded regions in any line oriented in the cross direction.

In some embodiments, in is convenient for the second layer further include a meltblown layer laminated to the continuous or discontinuous oriented monocomponent thermoplastic fibers. In some of these embodiments, the meltblown layer is disposed between two spunbonded layers.

### LISTING OF EXEMPLARY EMBODIMENTS

### Embodiment A

A method of forming a nonwoven fabric, comprising:

providing a first layer comprising continuous or discontinuous oriented monocomponent thermoplastic fibers;

providing a second layer;

conveying the first and second layers through a nip comprising a first and a second roller wherein the first roller is a patterned roller, wherein the nip pattern creates bonded regions while leaving unbonded regions so as to bond the first and second layers into a nonwoven fabric, wherein the unbonded regions comprise between about 20% and 40% of the total area of the nonwoven fabric, and further wherein the nip introduces latent heat into the nonwoven fabric; and

allowing the latent heat to cool while the first layer is held at a tension of less than 4 N/linear centimeter such that free loops are formed in the unbonded areas of the first layer.

### Embodiment B

The method according to Embodiment A wherein

the continuous or discontinuous oriented monocomponent thermoplastic fibers within the first layer are spunbond polypropylene formed at a spinning speed of lower than 2400 m/min.

### Embodiment C

The method according to Embodiments A or B wherein

the first and second layer comprise webs of indefinite length having a longitudinal direction and a cross direction perpendicular to the longitudinal direction, and wherein

no bonded regions extend without interruption by unbonded regions in any line oriented in the cross direction.

### Embodiment D

The method according to any of the above Embodiments wherein the bonded regions comprise between about 20% to 40% of the nonwoven fabric.

### Embodiment E

The method according to claim any of the above Embodiments wherein the second layer further comprises a meltblown layer laminated to the continuous or discontinuous oriented monocomponent thermoplastic fibers.

### Embodiment F

The method according to Embodiment E wherein the meltblown layer is disposed between two spunbonded layers.

### Embodiment G

A nonwoven fabric, comprising:

a first layer comprising continuous or discontinuous oriented uncrimped monocomponent thermoplastic fibers, and a second layer having a pattern of bonded regions and unbonded regions with the first layer,

wherein the bonded regions comprise between about 20% and 40% of the total area of the nonwoven fabric,

wherein the first layer displays raised loops in the unbonded areas, and

wherein the nonwoven fabric possesses a percent solidity of less than 12.

### Embodiment H

The nonwoven fabric according to Embodiment G wherein

the first and second layer comprise webs of indefinite length having a longitudinal direction and a cross direction perpendicular to the longitudinal direction, and wherein

no bonded regions extend without interruption by unbonded regions in any line oriented in the cross direction.

### Embodiment I

The nonwoven fabric according to Embodiments G or H wherein the bonded regions comprise between about 20% to 40% of the nonwoven fabric.

### Embodiment J

The nonwoven fabric according to claim any of Embodiments G through I wherein the second layer further comprises a meltblown layer laminated to the continuous or discontinuous oriented monocomponent thermoplastic fibers.

### Embodiment K

The nonwoven fabric according to Embodiment J wherein the meltblown layer is disposed between two spunbonded layers.

Various aspects and advantages of exemplary embodiments of the disclosure have been summarized. The above Summary is not intended to describe each illustrated embodiment or every implementation of the present certain exemplary embodiments of the present disclosure. The Drawings and the Detailed Description that follow more particularly exemplify certain preferred embodiments using the principles disclosed herein.

### BRIEF DESCRIPTION OF THE DRAWINGS

FIG. 1 is a perspective side view of an exemplary nonwoven fabric according to the present disclosure.

FIG. 2 is a schematic view of an apparatus for carrying out the method according to the present disclosure.

FIG. 3 is a schematic view of an alternate apparatus for carrying out the method.

In the drawings, like reference numerals indicate like elements. While the above-identified drawing, which may not be drawn to scale, sets forth various embodiments of the present disclosure, other embodiments are also contemplated, as noted in the Detailed Description. In all cases, this disclosure describes the presently disclosed disclosure by way of representation of exemplary embodiments and not by express limitations. It should be understood that numerous other modifications and embodiments can be devised by those skilled in the art, which fall within the scope and spirit of this disclosure.

### DETAILED DESCRIPTION

The present disclosure describes a nonwoven fabric particularly suited to be the loop side of a hook-and-loop fastening system. The disclosed method is simpler than other methods for making loop fabric, reducing cost for e.g. user of infant diapers.

For the following Glossary of defined terms, these definitions shall be applied for the entire application, unless a different definition is provided in the claims or elsewhere in the specification.

### Glossary

Certain terms are used throughout the description and the claims that, while for the most part are well known, may require some explanation. It should understood that, as used herein:

The terms “(co)polymer” or “(co)polymers” includes homopolymers and copolymers, as well as homopolymers or copolymers that may be formed in a miscible blend, e.g., by coextrusion or by reaction, including, e.g., transesterification. The term “copolymer” includes random, block and star (e.g. dendritic) copolymers.

The term “adjoining” with reference to a particular layer means joined with or attached to another layer, in a position wherein the two layers are either next to (i.e., adjacent to) and directly contacting each other, or contiguous with each other but not in direct contact (i.e., there are one or more additional layers intervening between the layers).

By using terms of orientation such as “atop”, “on”, “over,” “covering”, “uppermost”, “underlying” and the like for the location of various elements in the disclosed coated articles, we refer to the relative position of an element with respect to a horizontally-disposed, upwardly-facing substrate. However, unless otherwise indicated, it is not intended that the substrate or articles should have any particular orientation in space during or after manufacture.

The terms “about” or “approximately” with reference to a numerical value or a shape means+/−five percent of the numerical value or property or characteristic, but expressly includes the exact numerical value. For example, a viscosity of “about” 1 Pa-sec refers to a viscosity from 0.95 to 1.05 Pa-sec, but also expressly includes a viscosity of exactly 1 Pa-sec. Similarly, a perimeter that is “substantially square” is intended to describe a geometric shape having four lateral edges in which each lateral edge has a length which is from 95% to 105% of the length of any other lateral edge, but which also includes a geometric shape in which each lateral edge has exactly the same length.

The term “substantially” with reference to a property or characteristic means that the property or characteristic is exhibited to a greater extent than the opposite of that property or characteristic is exhibited. For example, a substrate that is “substantially” transparent refers to a substrate that transmits more radiation (e.g. visible light) than it fails to transmit (e.g. absorbs and reflects). Thus, a substrate that transmits more than 50% of the visible light incident upon its surface is substantially transparent, but a substrate that transmits 50% or less of the visible light incident upon its surface is not substantially transparent.

As used in this specification and the appended embodiments, the singular forms “a”, “an”, and “the” include plural referents unless the content clearly dictates otherwise. Thus, for example, reference to fine fibers containing “a compound” includes a mixture of two or more compounds. As used in this specification and the appended embodiments, the term “or” is generally employed in its sense including “and/or” unless the content clearly dictates otherwise.

As used in this specification, the recitation of numerical ranges by endpoints includes all numbers subsumed within that range (e.g. 1 to 5 includes 1, 1.5, 2, 2.75, 3, 3.8, 4, and 5).

Unless otherwise indicated, all numbers expressing quantities or ingredients, measurement of properties and so forth used in the specification and embodiments are to be understood as being modified in all instances by the term “about.” Accordingly, unless indicated to the contrary, the numerical parameters set forth in the foregoing specification and attached listing of embodiments can vary depending upon the desired properties sought to be obtained by those skilled in the art utilizing the teachings of the present disclosure. At the very least, and not as an attempt to limit the application of the doctrine of equivalents to the scope of the claimed embodiments, each numerical parameter should at least be construed in light of the number of reported significant digits and by applying ordinary rounding techniques.

Exemplary embodiments of the present disclosure may take on various modifications and alterations without departing from the spirit and scope of the present disclosure. Accordingly, it is to be understood that the embodiments of the present disclosure are not to be limited to the following described exemplary embodiments, but is to be controlled by the limitations set forth in the claims and any equivalents thereof.

### Exemplary Nonwoven Fabric, Apparatus and Processes

Various exemplary embodiments of the disclosure will now be described with particular reference to the Drawings.

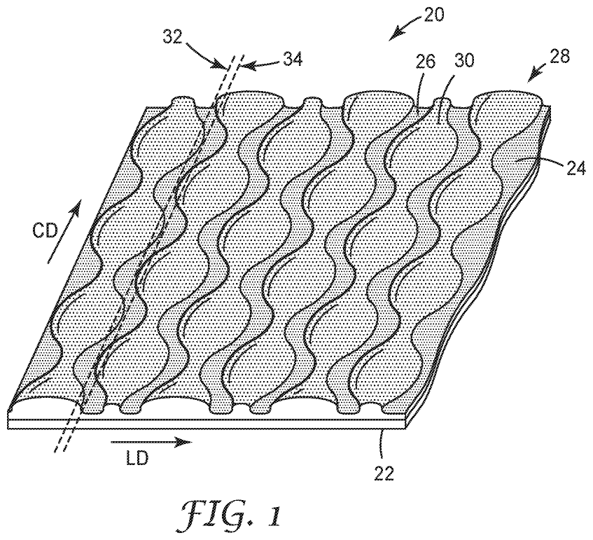

Referring now to FIG. 1, a perspective side view of a portion of an exemplary nonwoven fabric 20 according to the present disclosure is illustrated. Nonwoven fabric 20 includes a first layer 22 and a second layer 24. These layers comprise continuous or discontinuous oriented monocomponent thermoplastic fibers, but second layer 24 possesses increased stress induced crystallinity compared to first layer 22. First layer 22 and second layer 24 are bonded together at bond regions 26. Between bond regions 26, fibers within unbonded regions 28 from second layer 26 bulge upwards to provide loops 30.

In the depicted embodiment, nonwoven fabric 20 is a portion of a web of indefinite length with a longitudinal direction “LD” (also called the machine direction in the art) defined by the indefinite length. This further defines a cross direction “CD” perpendicular to the longitudinal direction and spanning the width of the web. In some convenient embodiments such as the one depicted, any arbitrary straight line drawn across the web in the cross direction (e.g. arbitrary lines 32 and 34) will pass through at least one unbonded region 28. In some convenient embodiments, bonded regions 26 cover between about 20% to 40% of the surface of nonwoven fabric 20.

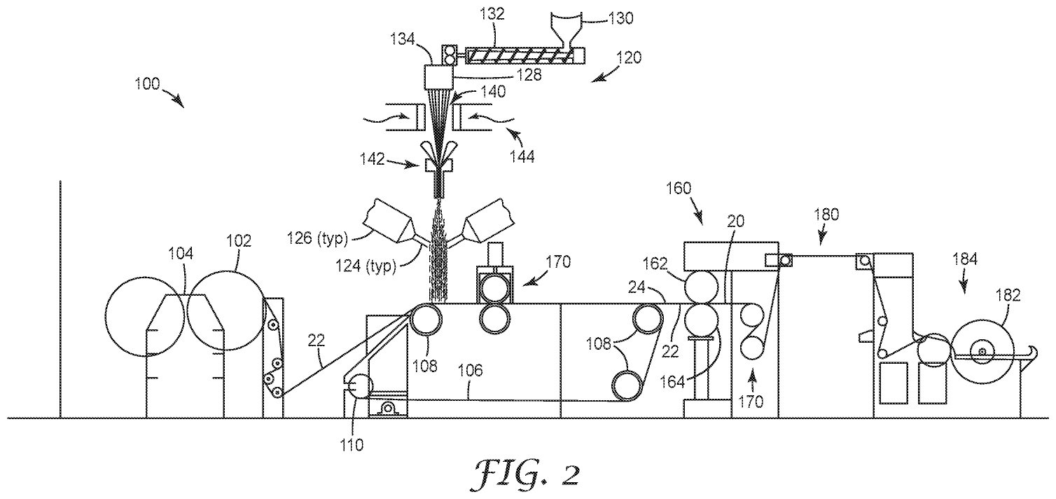

Referring now to FIG. 2, a schematic view of an apparatus 100 for carrying out the method according to the present disclosure is illustrated. In the illustrated embodiment of apparatus 100, first layer 22 in the form of a web of indefinite length is unwound from a roll 102 on an unwind stand 104. First layer 22 is directed towards a flexible belt 106 operation around idler rollers 108 and motor 110. Supported by flexible belt 106, first layer 22 passes under spinning station 120 where the fibers that will form second layer 24 are applied.

Spinning station 120 applies a stream 122 of continuous microfibers to first layer 22. In some embodiments stream 122 may optionally be supplemented by a secondary stream 124 of sub-micrometer fibers emanating from sub-micrometer fiber-forming apparatus 126.

In the depicted embodiment, fiber-forming material is brought to an extrusion head 128 from a hopper 130, feeding an extruder 132 where the material is melted. A pump 134 brings the molten material to extrusion pressure at extrusion head 128. Although solid polymeric material in pellet or other particulate form is most commonly used and melted to a liquid, pumpable state, other fiber-forming liquids such as polymer solutions can also be used.

Extrusion head 128 may be a conventional spinnerette or spin pack, generally including multiple orifices arranged in a regular pattern, e.g., straight-line rows. Filaments 140 of fiber-forming liquid are extruded from the extrusion head and conveyed to a processing chamber or optional attenuator 142. In some embodiments, quenching streams 144 of air or other gas are presented to filaments 140 to reduce their temperature of extruded filaments 140. It is considered within the scope of this disclosure, however for the streams of air or other gas to be heated to facilitate drawing of the fibers.

The filaments 140 then pass through the attenuator 142, and eventually exit onto first layer 22 where they are collected as a mass of fibers forming second layer 24. Additional information on the spinning of fibers in this way may be found in U.S. Pat. No. 8,906,815, “Composite nonwoven fibrous webs and methods of making and using the same,” Moore et al, which is hereby incorporated by reference as if rewritten. Optionally, the web with its first layer 22 and second layer 24 may be passed through pressing station 150 where a light nip is applied so that the layers will cling as they are conveyed along by flexible belt 106.

The web with its two layers are then conveyed to a bonding station 160, which in the depicted embodiment includes a patterned roll 162 and a smooth roll 164. At least one, and in many convenient embodiments both, of patterned roll 162 and smooth roll 164 are heated. Besides enough heat to form bond regions 26, enough latent heat must be instilled into the nonwoven fabric 20 to bring the first and second layers 22 and 24 above their T_{g}. It is desirable that nonwoven fabric 20 be kept at a low tension while the latent heat cools, so a tensioning station 170 is provided.

In the depicted embodiment, a cooling span 180 is provided to allow the latent heat from bond station 160 to cool slowly. During this time, the differential shrinkage of first layer 22 with second layer 24 causes loops 30 to form in unbonded regions 28. Finally, the nonwoven fabric 20 is wound onto a roll 182 on wind-up station 184.

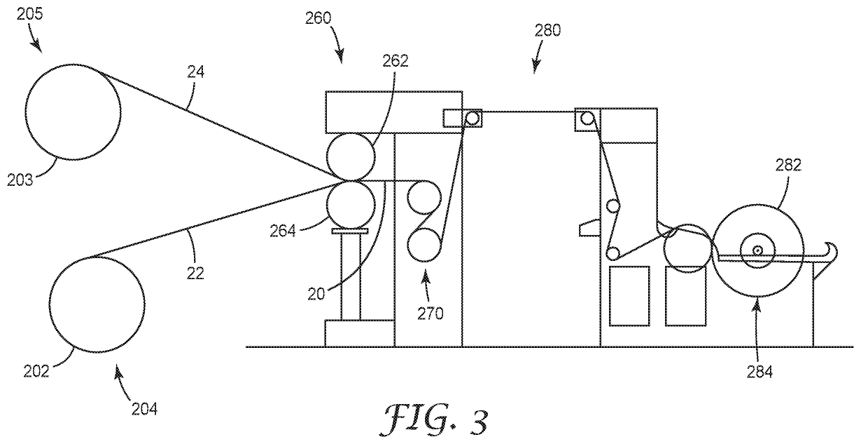

Referring now to FIG. 3, a schematic view of an alternate apparatus 200 for carrying out the method according to the present disclosure is illustrated. In the illustrated embodiment of apparatus 200, first layer 22 in the form of a web of indefinite length is unwound from a roll 202 on an unwind stand 204. Second layer 24, also in the form of a web of indefinite length is unwound from a roll 203 on an unwind stand 205. The two layers 22 and 24 are then conveyed to a bonding station 260, which in the depicted embodiment includes a patterned roll 262 and a smooth roll 264. At least one, and in many convenient embodiments both, of patterned roll 262 and smooth roll 264 are heated. Besides enough heat to form bond regions 26, enough latent heat must be instilled into the nonwoven fabric 20 to bring the first and second layers 22 and 24 above their T_{g}. It is desirable that nonwoven fabric 20 be kept at a low tension while the latent heat cools, so a tensioning station 270 is provided.

In the depicted embodiment, a cooling span 280 is provided to allow the latent heat from bond station 160 to cool slowly. During this time, the differential shrinkage of first layer 22 with second layer 24 causes loops 30 to form in unbonded regions 28. Finally, the nonwoven fabric 20 is wound onto a roll 282 on wind-up station 284.

The operation of exemplary embodiments of the present disclosure will be further described with regard to the following non-limiting detailed Examples. These examples are offered to further illustrate the various specific and preferred embodiments and techniques. It should be understood, however, that many variations and modifications may be made while remaining within the scope of the present disclosure.

### EXAMPLES

These Examples are merely for illustrative purposes and are not meant to be overly limiting on the scope of the appended claims. Notwithstanding that the numerical ranges and parameters setting forth the broad scope of the present disclosure are approximations, the numerical values set forth in the specific examples are reported as precisely as possible. Any numerical value, however, inherently contains certain errors necessarily resulting from the standard deviation found in their respective testing measurements. At the very least, and not as an attempt to limit the application of the doctrine of equivalents to the scope of the claims, each numerical parameter should at least be construed in light of the number of reported significant digits and by applying ordinary rounding techniques.

### Solidity Test

In the Examples below, percent solidity is determined by dividing the measured bulk density of a fibrous web by the density of the materials making up the solid portion of the web. Bulk density of a web can be determined by first measuring the weight (e.g. of a 10-cm by 10-cm section) of a web. Dividing the measured weight of the web by the web area provides the basis weight of the web, which is reported in g/m². Thickness of the web can be measured by obtaining (e.g., by die cutting) a 135 mm diameter disk of the web and measuring the web thickness with a 230 g weight of 100 mm diameter centered atop the web. The bulk density of the web is determined by dividing the basis weight of the web by the thickness of the web and is reported as g/m³. The solidity is then determined by dividing the bulk density of the web by the density of the material (e.g. polymer) comprising the solid fibers of the web. (The density of a polymer can be measured by standard means if the supplier does not specify material density.) Solidity is a dimensionless fraction which is reported as a percentage. This test also appears in U.S. Pat. No. 8,162,153, which is hereby incorporated by reference is if rewritten.

### Example 1

An apparatus was constructed generally as described in FIG. 2. A second layer was formed following the procedure of U.S. Pat. No. 8,906,815, “Composite nonwoven fibrous webs and methods of making and using the same,” except that the starting material was polypropylene commercially available as LUMICENE M6823MZ from Total of Courbevoie, FR, and the spinning speed was 3200 m/min. This material was then wound up and the wound roll was placed on the unwind stand of the apparatus.

From the unwind stand, the material was conveyed at a line speed of 139 ft/min (42.4 m/min) towards the spinning station. At the spinning station, LUMICENE M6823MZ polypropylene was spun bond onto the second layer with the spinning speed of the fibers being 1800 m/min, forming the first layer. The composite layers were then conveyed to the pressing station, where a light pressure of 300 pounds/linear inch (525 N/linear cm) was applied. The two layers were then conveyed to the bonding station where the patterned roll and the smooth roll were both heated to 260° F. (127° C.). The patterned roll had a pattern so as to form a bond pattern generally as depicted in FIG. 1. The bonded material was then conveyed to the tensioning station, where a tension regime of 2 pounds/linear inch (3.5 N/linear cm) was imposed, and loops were formed from the differential heating between the bonding zones and the non-bonding zones at the nip. The latent heat was slowly cooled over a cooling span of about 3 meters. At that point, the finished nonwoven fabric was wound up on a roll at a wind-up station. The finished fabric was then tested according to the Solidity Test, and a percent solidity of 11 was recorded.

### Example 2

A first layer was formed following the procedure of U.S. Pat. No. 8,906,815, except that the starting material was polypropylene commercially available as LUMICENE M6823MZ from Total of Courbevoie, FR, and the spinning speed was 790 m/min. This material was then wound up. A second layer was formed following the procedure of U.S. Pat. No. 8,906,815, except that the starting material was polypropylene commercially available as LUMICENE M6823MZ from Total of Courbevoie, FR, and the spinning speed was 2316 m/min. This material was then wound up.

The rolls of first and second layer materials were set upon the unwind stands of an apparatus constructed generally as depicted in FIG. 3. The two layers were then conveyed to the bonding station where the patterned roll and the smooth roll were both heated to 260° F. (127° C.). The patterned roll had a pattern so as to form a bond pattern generally as depicted in FIG. 1. The bonded material was then conveyed to the tensioning station, where a tension regime of 2 pounds/linear inch (3.5 N/linear cm) was imposed. The latent heat was slowly cooled over a cooling span of about 3 meters. During this time loops appeared in the second layer. At that point, the finished nonwoven fabric was wound up on a roll at a wind-up station. The finished fabric was then tested according to the Solidity Test, and a percent solidity of 11 was recorded.

### Example 3

An apparatus was constructed generally as described in FIG. 2. A second layer of a spunbond/meltblown/spunbond (SMS) composite, commercially available as S1202KR1BA01A from Fitesa of Simsonville, S.C., was placed on the unwind stand of the apparatus. From the unwind stand, the material was conveyed at a line speed of 139 ft/min (42.4 m/min) towards the spinning station. At the spinning station, LUMICENE M6823MZ polypropylene was spun bond onto the second layer with the spinning speed of the fibers being 1800 m/min, forming the first layer. The composite layers were then conveyed to the pressing station, where a light pressure of 300 pounds/linear inch (525 N/linear cm) was applied. The two layers were then conveyed to the bonding station where the patterned roll and the smooth roll were both heated to 260° F. (127° C.). The patterned roll had a pattern so as to form a bond pattern generally as depicted in FIG. 1. The bonded material was then conveyed to the tensioning station, where a tension regime of 2 pounds/linear inch (3.5 N/linear cm) was imposed. The latent heat was slowly cooled over a cooling span of about 3 meters. During this time, loops appeared in the second layer. At that point, the finished nonwoven fabric was wound up on a roll at a wind-up station. The finished fabric was then tested according to the Solidity Test, and a percent solidity of 10.8 was recorded

Reference throughout this specification to “one embodiment,” “certain embodiments,” “one or more embodiments” or “an embodiment,” whether or not including the term “exemplary” preceding the term “embodiment,” means that a particular feature, structure, material, or characteristic described in connection with the embodiment is included in at least one embodiment of the certain exemplary embodiments of the present disclosure. Thus the appearances of the phrases such as “in one or more embodiments,” “in certain embodiments,” “in one embodiment” or “in an embodiment” in various places throughout this specification are not necessarily referring to the same embodiment of the certain exemplary embodiments of the present disclosure. Furthermore, the particular features, structures, materials, or characteristics may be combined in any suitable manner in one or more embodiments.

While the specification has described in detail certain exemplary embodiments, it will be appreciated that those skilled in the art, upon attaining an understanding of the foregoing, may readily conceive of alterations to, variations of, and equivalents to these embodiments. Accordingly, it should be understood that this disclosure is not to be unduly limited to the illustrative embodiments set forth hereinabove. In particular, as used herein, the recitation of numerical ranges by endpoints is intended to include all numbers subsumed within that range (e.g., 1 to 5 includes 1, 1.5, 2, 2.75, 3, 3.80, 4, and 5). In addition, all numbers used herein are assumed to be modified by the term “about.”

Furthermore, all publications and patents referenced herein are incorporated by reference in their entirety to the same extent as if each individual publication or patent was specifically and individually indicated to be incorporated by reference. Various exemplary embodiments have been described. These and other embodiments are within the scope of the following claims.

1. A method of forming a nonwoven fabric, comprising:
providing a first layer comprising continuous or discontinuous oriented monocomponent thermoplastic fibers;
providing a second layer;
conveying the first and second layers through a nip comprising a first and a second roller wherein the first roller is a patterned roller, wherein the nip pattern creates bonded regions while leaving unbonded regions so as to bond the first and second layers into a nonwoven fabric, wherein the unbonded regions comprise between about 20% and 40% of the total area of the nonwoven fabric, and further wherein the nip introduces latent heat into the nonwoven fabric; and
allowing the latent heat to cool while the first layer is held at a tension of less than 4 N/linear centimeter such that free loops are formed in the unbonded areas of the first layer.

2. The method according to claim 1 wherein
the continuous or discontinuous oriented monocomponent thermoplastic fibers within the first layer are spunbond polypropylene formed at a spinning speed of lower than 2400 m/min.

3. The method according to claim 1 wherein
the first and second layer comprise webs of indefinite length having a longitudinal direction and a cross direction perpendicular to the longitudinal direction, and wherein
no bonded regions extend without interruption by unbonded regions in any line oriented in the cross direction.

4. The method according to claim 1 wherein the bonded regions comprise between about 20% to 40% of the nonwoven fabric.

5. The method according to claim 1 wherein the second layer further comprises a meltblown layer laminated to the continuous or discontinuous oriented monocomponent thermoplastic fibers.

6. The method according to claim 5 wherein the meltblown layer is disposed between two spunbonded layers.

7. A nonwoven fabric, comprising:
a first layer comprising continuous or discontinuous oriented uncrimped monocomponent thermoplastic fibers, and a second layer having a pattern of bonded regions and unbonded regions with the first layer,
wherein the bonded regions comprise between about 20% and 40% of the total area of the nonwoven fabric,
wherein the first layer displays raised loops in the unbonded areas, and
wherein the nonwoven fabric possesses a percent solidity of less than 12.

8. The nonwoven fabric according to claim 7 wherein
the first and second layer comprise webs of indefinite length having a longitudinal direction and a cross direction perpendicular to the longitudinal direction, and wherein
no bonded regions extend without interruption by unbonded regions in any line oriented in the cross direction.

9. The nonwoven fabric according to claim 7 wherein the bonded regions comprise between about 20% to 40% of the nonwoven fabric.

10. The nonwoven fabric according to claim 7 wherein the second layer further comprises a meltblown layer laminated to the continuous or discontinuous oriented monocomponent thermoplastic fibers.

11. The nonwoven fabric according to claim 10 wherein the meltblown layer is disposed between two spunbonded layers.
